(19) United States
(12) Patent Application Publication
(10) Pub. No.: US 2023/0130124 A1
(43) Pub. Date: Apr. 27, 2023

(54) COSMETIC MICRONEEDLE

(71) Applicant: COSMED PHARMACEUTICAL CO., LTD., Kyoto-city (JP)

(72) Inventors: Ying-shu Quan, Kyoto-city (JP); Fumio Kamiyama, Kyoto-city (JP)

(21) Appl. No.: 17/452,216

(22) Filed: Oct. 25, 2021

(51) Int. Cl. A61K 8/02; A61K 8/24; A61Q 19/08; A61K 8/73; A61K 8/86

(52) U.S. Cl. CPC A61K 8/0216; A61K 8/24; A61Q 19/08; A61K 8/735; A61K 8/86

(57) ABSTRACT

To provide a beauty care sheet that can be realized much more easily than conventional methods in terms of a device and an effective manner for the purpose of filling groove portions formed into fine wrinkles, sagging wrinkles and the like of the skin and recovering youthful swelling of the skin. A microneedle array for skin application having a water-soluble polymer as a base and hydroxyapatite particles dispersed in the base, a beauty sheet intended to care for a groove portion of the skin, including the microneedle array for skin application, and a beauty sheet intended to care for wrinkles between eyebrows, wrinkles of nasolabial folds, or an intermammary groove, including the microneedle array for skin application. The particle size of the hydroxyapatite particles is preferably 5 μm or less.

### BACKGROUND OF THE INVENTION

### Field of the Invention

The present invention relates to a technical field of microneedles applied to the skin. Specifically, the present invention relates to a microneedle array for administration of a composition containing hydroxyapatite which is easy to fix collagen and is effective for skin augmentation by proliferation.

### Description of the Related Art

Skin is composed of epidermis and dermis. The outermost epidermis is formed by stratified squamous epithelium and underlying basement membrane. The epidermis does not contain blood vessels and is grown by a diffusion action from the dermis. The epidermis is mainly composed of keratinocytes, and there are also black cells and Langerhans cells. This skin layer acts as a barrier between the body and the external environment, keeping water inside the body and preventing entry of harmful chemicals and pathogens.

The dermis is beneath the epidermis and includes a plurality of structures including blood vessels, nerves, hair follicles, smooth muscle, glands, and lymphatic tissue. The dermis (or corium) is typically 0.1 to 3 mm thick and is a main component of human skin. The dermis is composed of connective tissue of a network structure, predominantly present and supporting collagen fibrils, and elastin fibers providing flexibility. Main cell types that make up the dermis are fibroblasts, adipocytes (fat reservoirs), and macrophages.

The hypodermis is beneath the dermis and is important for attaching the skin to lower bones and muscles and for supplying blood vessels and nerves to the skin. The hypodermis is generated in loose connective tissue and contains fibroblasts, macrophages, and adipocytes. Adipocytes play a major role in fat storage function of the hypodermis. Fat serves as a filling material and as an insulator of the body from the external environment.

Facial aging occurs as a result of a plurality of factors, among which changes in the skin that it inherently possesses, action of gravity, activation of facial muscles that produce mechanical facial expressions, skin loss or migration, osteopenia, reduced tissue elasticity, and harsh environmental conditions, in particular, sunlight or ultraviolet radiation and exposure to pollutants. The skin ages as the epidermis begins to become thinner and its junction with the dermis becomes flattened. in response to human aging, collagen is reduced, and collagen fiber bundles that give skin turgor are loosened and reduce strength. When the skin loses elasticity, it becomes less able to withstand tension. With gravity, muscle damage and tissue changes, the skin begins to form fine wrinkles. Lack of water and disruption of intercellular binding can also reduce skin barrier function and increase skin pore size.

Efforts have been made to develop and use compositions that repair skin defects such as fine wrinkles, or compositions that increase tissue of a subject, in order to improve appearance of the skin, particularly facial skin.

Currently, there are numerous known dermal fillers for skin augmentation, such as autologous implantable materials, allogeneic products, xenogeneic products, and synthetic products. Available dermal fillers include biodegradable natural substances (such as collagen, gelatin, hyaluronic acid, and dextran), biodegradable synthetic polymers (such as poly-L-lactic acid and carboxymethyl cellulose), non-biodegradable synthetic polymers (such as silicones), and combinations thereof.

Biocompatible ceramic skin augmentation materials such as hydroxyapatite are known to be efficient skin augmentation materials due to their nature.

Hydroxyapatite (Ca₅(PO₄)₃(OH)) is a naturally occurring mineral form of calcium phosphate. Hydroxyapatite contains a mineral component of bone and thus becomes biocompatible and non-immunogenic when introduced into the body of a subject. When injected as small microspheres, hydroxyapatite acts as a scaffold that promotes new tissue formation similar to its surrounding environment. It is known that inside skin such as the dermis, attached hydroxyapatite particles support fibroblast ingrowth and. new collagen formation.

There is a microneedle as a measure for administering hydroxyapatite intradermally without injection or surgery. Patent Document 1 discloses a device and a method for delivering a dermal filler composition to the skin of a subject using a hollow type microneedle device. The present disclosure is a miniaturized version of a conventional injection method.

Patent Document 2 discloses a device in which hydroxyapatite is loaded into a microneedle with an applicator and inserted into the skin. This content is intended to deliver hydroxyapatite with a particle size of 5 microns or more intradermally by an applicator microneedle integrated system having a complicated configuration.

### PRIOR ART DOCUMENT

### Patent Document

Patent Document 1: WO 2008/072229 A2

Patent Document 2: JP 6546091 B

### SUMMARY OF THE INVENTION

### Problems to be Solved by the Invention

An object of the present invention is to fill groove portions formed in fine wrinkles, sagging wrinkles and the like of the skin and recover youthful swelling of the skin, and to provide a beauty care sheet that can be realized much more easily than conventional methods in terms of a device and an effective manner for this purpose.

### Means for Solving the Problems

The present inventors have succeeded. in producing a microneedle array in which a substance soluble in the body is selected as a base of a microneedle, a particle diameter of hydroxyapatite is adjusted within a predetermined range, and hydroxyapatite is dispersed in the base, conducted a test using the microneedle array, and found that the desired object is achieved, thereby completing the present invention.

The present invention is as described below.

[1] A microneedle array for skin application including a water-soluble polymer as a base and hydroxyapatite particles dispersed in the base.

[2] A beauty sheet intended to care for a groove portion of the skin, including a microneedle array for skin application having a water-soluble polymer as a base and hydroxyapatite particles dispersed in the base.

[3] A beauty sheet intended to care for wrinkles between eyebrows, wrinkles of nasolabial folds, or an intermammary groove, including a microneedle array for skin application having a water-soluble polymer as a base and hydroxyapatite particles dispersed in the base.

[4] The microneedle array according to [1] or the beauty sheet according to [2] or [3], wherein the hydroxyapatite particles have a particle diameter of 5 μm or less.

[5] The microneedle array or the beauty sheet according to [4], wherein the hydroxyapatite particles have a particle diameter of 1 μm or less.

[6] The microneedle array or the beauty sheet according to any one of [1] to [5], wherein a content of the hydroxyapatite particles in the base is 0.0001% by mass or more and 40% by mass or less.

[7] The microneedle array or the beauty sheet according to any one of [1] to [5], wherein a content of hydroxyapatite particles in the base is 0.001% by mass or more and 30% by mass or less.

[8] The microneedle array or the beauty sheet according to any one of [1] to [7], wherein a needle size of the microneedle is 0.1 mm or more and 1.0 mm or less.

[9] The microneedle array or the beauty sheet according to any one of [1] to [8], wherein the water-soluble polymer is one kind or two or more kinds selected from the group consisting of sodium hyaluronate and derivatives thereof, collagen, proteoglycan, hydroxypropyl cellulose, chondroitin sulfate, carboxymethyl cellulose, polyvinyl pyrrolidone, polyethylene glycol, polyvinyl alcohol (PVA), and dextran.

[10] The microneedle array or the beauty sheet according to [9], wherein the sodium hyaluronate and derivatives thereof are PEG-grafted sodium hyaluronate, and hydroxyapatite fine particles are dispersed in the PEG-grafted sodium hyaluronate.

The microneedle array or the beauty sheet according to any one of [1] to [10], wherein hydroxyapatite fine particles are dispersed in the base by a surfactant.

[12] The microneedle array or the beauty sheet according to [11], wherein the surfactant is a POE (20) oleyl ether.

[13 ] The microneedle array or the beauty sheet according to any one of [1] to [12], in which the microneedle array is a substrate having a thickness of 100 μm or less and has a flexible substrate portion.

[14 ] The microneedle array or the beauty sheet according to any one of [1] to [13], wherein the base of the microneedle array contains 2% by mass or more of a water-soluble low-molecular compound in addition to the water-soluble polymer.

[15] The microneedle array or the beauty sheet according to any one of [1] to [14], wherein a back surface of the microneedle array is lined with a hydrophobic or undissolved film.

The present invention relates to a microneedle array containing particulate hydroxyapatite (hereinafter referred to as HAP) as a skin augmentation composition. Specifically, the microneedle array of the present invention has a needle length. of 1 mm or less in which fine particles HAP of 5 μm or less are uniformly dispersed in an intradermal soluble base, and is useful for filling undesirable fine wrinkles and sagging wrinkles of the skin of a subject to be dissolved. According to the present invention, the microneedle injects the HAP fine particles in the intradermal soluble base periodically into an epidermal layer and a dermal layer of the skin of the subject, thus accumulating there for a long period of time and continuing to induce filling and collagen proliferation effects.

### BRIEF DESCRIPTION OF THE DRAWINGS

FIG. 1 is a schematic view showing a use period and a measurement schedule in an efficacy test of a microneedle array of the present invention.

### DETAILED DESCRIPTION OF THE PREFERRED EMBODIMENTS

The microneedle array of the present invention is a microneedle array in which fine particles HAP are uniformly dispersed in an intradermal soluble base, includes a substrate and a plurality of microneedles on the substrate, and is integrally formed using a water-soluble polymer as a base.

The beauty sheet of the present invention may be a beauty sheet intended to care for a groove portion of the skin, or may be a beauty sheet intended to care for wrinkles between eyebrows, wrinkles of nasolabial folds, or an intermammary groove.

Base of microneedle array (or beauty sheet)

The base of the microneedle array is a water-soluble polymer. When a microneedle array uniformly containing HAP is prepared using such a material by a conventional method, HAP is contained not only in a microneedle portion but also in a substrate portion. Such a microneedle array may be used, or a microneedle array not containing the HAP in a substrate portion may be used. When the microneedle array is applied to a site where it is desired to improve appearance of the facial skin, the microneedle portion can reach the inside of the epidermis and the inside of the dermis, and the microneedle base is dissolved in the skin to enable delivery of the contained HAP to a target site.

Examples of the water-soluble polymer include sodium hyaluronate and derivatives thereof (for example, polyethylene oxide grafted sodium hyaluronate, also referred to as PEG-grafted sodium hyaluronate), collagen, proteoglycan, hydroxypropyl cellulose, chondroitin sulfate, carboxymethyl cellulose, polyvinylpyrrolidone, polyethylene glycol, polyvinyl alcohol (PVA), dextran, and the like, and one kind or two or more kinds selected therefrom may be used in mixture. In particular, sodium hyaluronate or a derivative thereof is preferable.

Hyaluronic acid is typically a sodium salt, but is a type of glycosaminoglycan (mucopolysaccharide), and has a structure in which disaccharide units of N-acetylglucosamine and glucuronic acid are linked. Examples of the hyaluronic acid include biologically derived hyaluronic acid isolated from cockscombs, umbilical cords and the like, culture-derived hyaluronic acid mass-produced by lactic acid bacteria, streptococci and the like, and the like. The biologically derived hyaluronic acid cannot completely remove collagen of the organism from which the biologically derived hyaluronic acid is derived, and the remaining collagen may exert an adverse effect. Therefore, a culture-derived hyaluronic acid which does not contain collagen is preferred. Therefore, hyaluronic acid preferably contains 50% by mass or more of culture-derived hyaluronic acid.

In preparing a microneedle array using a water-soluble polymer substance selected from sodium hyaluronate or a derivative thereof as a component, when the weight average molecular weight decreases, the microneedle array molded from these polymer substances tends to be hard and easily stick in an application site. Conversely, when the weight average molecular weight increases, the microneedle array tends to be flexible and easily applied to bending of gums and the like since mechanical strength is improved and the microneedle array becomes sticky. For the purpose of the present invention, the weight average molecular weight is preferably 5,000 to 2,000,000.

When the microneedle array is applied to the skin, the microneedle array may be formed from a mixture of a high-molecular-weight polymer substance having a weight average molecular weight of 100,000 or more and a low-molecular-weight polymer substance having a weight average molecular weight of 50,000 or less because the microneedle array has moderate hardness and is hardly broken. The weight average molecular weight of the high-molecular-weight polymer substance may be 50,000 or more, and is preferably 2,000,000 or less. Also, the weight average molecular weight of the low-molecular-weight polymer substance may be 50,000 or less, and is preferably 1,000 or more. In the present invention, the weight average molecular weight is a value measured by gel permeation chromatography (GPO).

The ratio at the time of mixing the high-molecular-weight polymer substance and the low-molecular-weight polymer substance varies also depending on the type and weight average molecular weight of each polymer substance. Therefore, the ratio may be appropriately determined so as to have preferable mechanical strength and hardness, but in general, it is preferably 1% by mass or more of the high-molecular-weight polymer substance and 99% by mass or less of the low-molecular-weight polymer substance.

PEG-Grafted sodium hyaluronate is described in International Application No. 2017/010518 and can also be suitably used in the present invention.

In order to promote dissolution of the microneedle in the skin, a soluble agent may be added to the polymer substance. Examples of the soluble agent include monosaccharides such as trehalose and glucose, disaccharides, polyhydric alcohols such as glycerin, propylene glycol (PG), butylene glycol (BG) and polyethylene glycol (PEG), and the like. An addition amount of the soluble agent is desirably 1% by mass or more and 50% by mass or less as a concentration in the base.

The base of the microneedle array can contain a water-soluble low-molecular compound in addition to the water-soluble polymer. The water-soluble low-molecular compound is a monosaccharide, a disaccharide, or a polyhydric alcohol used as the above-mentioned soluble agent, and is a compound having a molecular weight of 500 or less. Examples of the monosaccharide include glucose, fructose, and the like, and examples of the disaccharide include sucrose, lactose, trehalose, maltose, and the like. Examples of the polyhydric alcohol include glycerin, propylene glycol (PG), butylene glycol (BG), polyethylene glycol (PEG) 200, PEG400, and the like.

The addition amount of the water-soluble low-molecular compound is 0.1% by mass or more and 50% by mass or less, preferably 0.5% by mass or more and 40% by mass or less, and more preferably 1% by mass or more and 30% by mass or less as a concentration in the base.

In order to uniformly disperse HAP in the water-soluble base, a surfactant may be added. As the surfactant, a nonionic surfactant is preferred. In particular, a water-soluble surfactant is more preferable. The addition amount is 1:0.001 to 1:100 based on the mass of HAP. As the surfactant of a microneedle coating solution of the present invention, a polyoxyethylene sorbitan monofunctional fatty acid ester such as polysorbate 80, polysorbate 40 or polysorbate 20, a polyoxyethylene alkyl ether such as polyoxyethylene (5) cetyl ether or polyoxyethylene (20) cetyl ether, or a mixture thereof can be suitably used.

Shape of microneedle array

The height of the microneedle is desirably 50 μm or more and 1,000 μm or less, and more preferably 100 μm or more and 250 μm or less. When the height is less than 50 pm, it is disadvantageous for delivery of a local anesthetic agent. When the height exceeds 1,000 μm, pain or bleeding may be accompanied at the time of application.

A tip of the microneedle is desirably a circle having a diameter of 1 μm or more and a diameter of 50 μm or less or a flat surface having the same area. Within this range, it is advantageous for intradermal delivery of the microneedle. Examples of the needle shape include a rod shape, a truncated cone shape and Konide, and a truncated cone shape or a Konide shape is desirable.

The thickness of the substrate of the microneedle array is desirably 5 μm or more and 100 μm or less, and more preferably 10 μm or more and 60 μm or less.

The shape of the substrate of the microneedle array can be appropriately set according to the application site, and examples thereof include a circle, an ellipse, a triangle, a quadrangle, a polygon, and the like. The size of the shape is usually 2 mm or more and 200 mm or less, and preferably 5 mm or more and 100 mm or less, in terms of diameter (long diameter) or length of one side (long side). In addition, the size of the microneedle array is usually 5 mm² or more and 2000 mm² or less, and preferably 10 mm² or more and 1000 mm² or less, in terms of area.

HAP

An active ingredient contained in the microneedle array of the present invention is HAP. HAP Particles delivered into the skin promote fibroblast ingrowth and new collagen formation. Since the promotion of collagen formation reacts from the surface of HAP particles, effects of promoting collagen formation and reducing wrinkles of HAP vary depending on the particle size of HAP even when the ame amount of HAP is delivered into the skin. That is, the particle size is desirably small. The particle size of HAP of the present invention is desirably 5 μm or less, more desirably 2 μm or less, and further desirably 1 μm or less. Although the collagen forming effect is obtained when the particle size of HAP is 5 μm or more, the surface area per the same weight is small, and thus the collagen forming effect is also small. In addition, since the tip portion of the microneedle has a diameter of 1 μm or more or is a circular plane, it is also disadvantageous to form the needle when the particle diameter of HAP is 5 μm or more.

In addition to the HAP, additives usually contained in pharmaceuticals and cosmetics may be contained. A concentration of an additive contained in the microneedle array of the present invention can be set in an appropriate range according to the type of the additive, the purpose of addition, and the like.

A concentration of HAP in the base is 0.0001% by mass or more and 40% by mass or less, and more preferably 0.001% by mass or more and 30% by mass or less. Here, the concentration of HAP in the base is a mass in total weight of the microneedle array. When the concentration of HAP in the base is 0.0001% by mass or less, it is difficult to exhibit the skin anti-wrinkle effect, and when the concentration of HAP in the base is 40% by mass or more, the microneedle becomes brittle, and difficulty may occur in skin insertion.

The method for producing the microneedle array of the present invention is not particularly limited, and may be produced by any conventionally known method, and examples thereof include a method in which an aqueous solution containing the water-soluble polymer and HAP, and if necessary, other components is cast in a mold in which the shape of the microneedle is bored, dried, and then peeled off. The peeled microneedle array sheet is cut and used according to the shape of the application site of the skin.

The microneedle array of the present invention is conveniently used as a microneedle patch below.

Microneedle patch

The microneedle patch of the present invention includes the microneedle array, and a support provided on a back surface of the microneedle array. Here, the back surface of the microneedle array is a substrate portion opposite to the surface from which the microneedles protrude. The support is not essential, but the microneedle patch is easy to handle with the support, and the support can prevent slipping from the application site. A microneedle patch having a back surface of a microneedle array lined with a hydrophobic or undissolved film as a support is an embodiment of a cosmetic anti-wrinkle formulation.

The microneedle patch can be applied to the skin, and rapidly dissolved with a liquid such as water or a lotion, an emulsion or a serum from the back surface of the microneedle patch.

A formulation dosage form in the present invention can have various forms. These will be sequentially described.

1. A microneedie patch in which a polymer film is lined as a support on a back surface of a dried microneedle array produced by the method for producing a microneedie array. The present formulation is integrated with the polymer film on the back surface of the microneedie array with an adhesive or a pressure-sensitive adhesive. The size of the microneedle array and the polymer film may be processed such that the polymer film has skin adhesiveness on larger film surface. The polymer film may be water permeable such as porous or woven fabric. Typical examples thereof include plastic sheets or films of polyethylene, polypropylene, polyethylene terephthalate, ethylene vinyl acetate copolymer (EVA) and the like; paper sheets such as sterilized paper, cellophane, nonwoven fabric, and woven fabric; and the like.

The size of the support is preferably larger than that of the microneedle array, in order to reinforce adhesive force of the microneedle array on the skin from the back surface. The support can be set to a size and shape that are easy to handle according to the application site, and for example, it is appropriate to make the support about 3 to 20 mm larger from the outer edge of the microneedle array. The thickness of the support may be equal to, thicker or thinner than the thickness of the microneedle array substrate, and can be appropriately set to a thickness that can support a flexible and thin microneedle array and is easy to handle.

The support desirably has skin adhesiveness, in order to reinforce the adhesive force of the microneedle array on the skin from the back surface.

One aspect for ensuring the skin adhesiveness of the support includes a support in which an adhesive substance is coated on the support, that is, a pressure-sensitive adhesive is applied to the support. Here, examples of the adhesive substance include pressure-sensitive adhesives usually used in patch preparations, and for example, grades having wet surface adhesiveness of acrylic, silicon-based, and rubber-based pressure-sensitive adhesives are preferred.

The microneedle patch of the present invention can be produced by covering the back surface of the microneedle array with a support.

### EXAMPLES

Hereinafter, the present invention will be described by exemplifying Examples, but the present invention is not limited to Examples.

### Example 1

Hydroxyapatite (average particle diameter=2 μm) (purchased from Wakenyaku Co., Ltd.) (2.5 parts by mass) and 50 parts by mass of sodium hyaluronate (FCH-SU, Kikkoman Corporation) were weighed, and water was added thereto to prepare a solution with a solid content of 10% by mass. The aqueous solution was poured into a mold of a Konide type having a needle length of 200 μm and having a microneedle substrate diameter of 10 mm, dried at room temperature for 24 hours, and punched to produce a microneedle array. Thereafter, a polyethylene (PE) adhesive film was adhered to the back surface of the array (microneedle substrate thickness=40 to 50 μm, support thickness=40 to 60 μm).

### Examples 2 to 7, Comparative Examples 1 to 3

Microneedle formulations containing the base and hydroxyapatite described in Table 2 were produced according to the method described in Example 1.

As a comparative example, a gel ointment (Comparative Example 2) formulation was produced based on the composition in Table 1.

### Test Example 1

Summary of efficacy test

1) Sample

### Examples 1, 2 and Comparative Examples 2, 3

2) Application Site

Below both eyes of panelists

3) Methods of Use

After face washing, the face was conditioned with basic cosmetics, and after confirming that no moisture and oil remained in the face, the sample was applied to each site before going to bed, and peeled the next morning.

4) Application Frequency

Each sample was used every other day for 4 weeks. Thereafter, in order to stop the use and confirm persistence of the effect, the skin was measured 8 weeks after the start.

5) Number of Panelists

4 panelists

6) Measurement Item

Replica analysis of wrinkles around eyes (area)

7) Measurement Time/Schedule

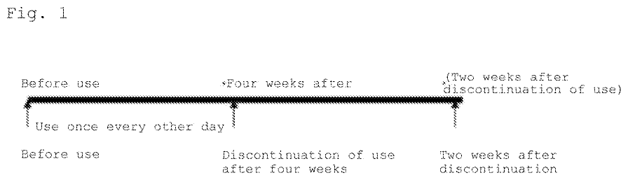

The outline is shown in FIG. 1.

<Measurement Method>

Wrinkles around the eye were analyzed with a three-dimensional image analysis system Primos lite 4530 (GFMesstechnik GmbH).

The area % of wrinkle part was analyzed by image analysis. Results are shown in Table 1.

TABLE 1

| Example | | | | Wrinkle area ratio (%) | | |
|---|---|---|---|---|---|---|
| Comparative | HAP % | | Backing | Before | Four weeks | Two weeks after |
| Example | in Base | Base | agent | use | after | discontinuation |
| Example 1 | HAP 10% | Na hyaluronate | Polyethylene | 5.4 | 3.8 | 3.4 |
| | Average particle | | adhesive film | | | |
| | diameter = 2 μm | | | | | |
| Example 2 | HAP 10% | Na hyaluronate 70% | Polyethylene | 5.2 | 3.5 | 2.9 |
| | Average particle | Glucose 30% | adhesive film | | | |
| | diameter = 1 μm | | | | | |
| Comparative | HAP None | Na hyaluronate | Polyethylene | 5.2 | 4.2 | 4.6 |
| Example 1 | | | adhesive film | | | |
| No HAP | | | | | | |
| MN | | | | | | |
| Comparative | HAP 10% | Macrogol | Polyethylene | 5.3 | 5.2 | 5.2 |
| Example 2 | Average particle | Perfume | adhesive film | | | |
| Gel ointment | diameter = 1 μm | | | | | |
| Reference | HAP 10% | Na hyaluronate 70% | Polyethylene | 5.4 | 4.3 | 4.9 |
| Example 1 | Average particle | Glucose 30% | adhesive film | | | |
| HAP Large | diameter = 20 μm | | | | | |
| particle diameter | | | | | | |
| MN | | | | | | |

In Comparative Example 1 and Reference Example 1, the wrinkle reduction effect was observed after 4 weeks of use probably due to hyaluronic acid rather than the effect of HAP. From Examples 1 and 2, it is determined that the effect of HAP is sustained even after the lapse of 2 weeks after the discontinuation.

### Test Example 2

HAP in a base solution for microneedle molding (5 to 10% by mass water-soluble polymer aqueous solution) was added, the mixture was stirred at high speed, and left to stand for 15 minutes, then HAP dispersibility in the base solution was confirmed by presence or absence of precipitation of HAP. The number of needles in the molded microneedle patch that could be normally molded was counted to obtain a molding rate. Further, brittleness (evaluated based on whether toughness was sufficient, specifically, whether the patch was broken by bending the patch 90 degrees) of the patch was confirmed. Results are shown in Table 2.

TABLE 2

| | | | Particles of HAP | | | |
|---|---|---|---|---|---|---|
| | | | Blending amount | Dispersibility of HAP | Microneedle | Toughness |
| | Base | Surfactant | (% in base) | in base solution | molding rate | of patch |
| Example 3 | Sodium hyaluronate | — | 1 μm or less | No HAP precipitation | 100% | ◯ |
| | | | (0.5%) | | | |
| Example 4 | Sodium hyaluronate | — | 1 μm or less | No HAP precipitation | 100% | ◯ |
| | | | (1%) | | | |
| Example 5 | PEG-Grafted sodium | — | 0.2 μm or less | No HAP precipitation | 100% | ◯ |
| | hyaluronate | | (0.1%) | | | |
| Example 6 | PEG-Grafted sodium | POE (20) | 0.2 μm or less | No HAP precipitation | 100% | ◯ |
| | hyaluronate | Cetyl ether 3% | (10%) | | | |
| Example 7 | PEG-Grafted sodium | Hydrogenated lecithin | 0.2 μm or less | No HAP precipitation | 100% | ◯ |
| | hyaluronate | 0.001% | (3%) | | | |
| Reference | Sodium hyaluronate | — | 100 to 150 μm | HAP Precipitation | 0% | — |
| Example 2 | | | (0.5%) | | | |
| Reference | Sodium hyaluronate | POE (20) | 1 μm or less | HAP Precipitation | 60% | X |
| Example 3 | | Cetyl ether 10% | (45%) | | | |

% represents % by mass.

From Table 2, as shown in Examples 3 to 7, microneedle moldability is good and the toughness is also sufficient. From the reference examples, it is found that when the particle size of HAP is too large, molding of the HAP-containing microneedle is impossible, and even when the particle size is small, the microneedle becomes brittle when the HAP content is large.

What is claimed is:

1. A microneedle array for skin application in which a water-soluble polymer is used as a base and hydroxyapatite particles are dispersed in the base.

2. A beauty sheet intended to care for a groove portion of the skin, comprising a microneedle array for skin application having a water-soluble polymer as a base and hydroxyapatite particles dispersed in the base.

3. A beauty sheet intended to care for wrinkles between eyebrows, wrinkles of nasolabial folds, or an intermammary groove, comprising a microneedle array for skin application having a water-soluble polymer as a base and hydroxyapatite particles dispersed in the base.

4. The microneedle array according to claim 1, in which the hydroxyapatite particles have a particle size of 5 μm or less.

5. The microneedle array according to claim 4, in which the hydroxyapatite particles have a particle size of 1 μm or less.

6. The microneedle array according to claim 1, in which a content of the hydroxyapatite particles in the base is 0.0001% by mass or more and 40% by mass or less.

7. The microneedle array according to claim 1, in which a content of hydroxyapatite particles in the base is 0.001% by mass or more and 30% by mass or less.

8. The microneedle array according to claim 1, in which a needle size of the microneedle is 0.1 mm or more and 1.0 mm or less.

9. The microneedle array according to claim 1, in which the water-soluble polymer is one kind or two or more kinds selected from the group consisting of sodium hyaluronate and derivatives thereof, collagen, proteoglycan, hydroxypropyl cellulose, chondroitin sulfate, carboxymethyl cellulose, polyvinyl pyrrolidone, polyethylene glycol, polyvinyl alcohol (PVA), and dextran.

10. The microneedle array according to claim 9, in which the sodium hyaluronate and derivatives thereof are PEG-grafted sodium hyaluronate, and hydroxyapatite fine particles are dispersed in the PEG-grafted sodium hyaluronate.

11. The microneedle array according to claim 1, in which hydroxyapatite fine particles are dispersed in the base by a surfactant.

12. The microneedle array according to claim 11, in which the surfactant is a POE (20) oleyl ether.

13. The microneedle array according to claim 1, in which the microneedle array is a substrate with a thickness of 100 μm or less and has a flexible substrate portion.

14. The microneedle array according to claim 1, in which the base of the microneedle array contains 2% by mass or more of a water-soluble low-molecular compound in addition to the water-soluble polymer.

15. The microneedle array according to claim 1, in which a back surface of the microneedle array is lined with a hydrophobic or undissolved film.
